(19) United States
(12) Patent Application Publication
(10) Pub. No.: US 2022/0177504 A1
(43) Pub. Date: Jun. 9, 2022

(54) PLATINUM COMPLEXES HAVING BINAPHTHYL-BASED DIPHOSPHINE LIGANDS FOR THE SELECTIVE CATALYSIS OF THE HYDROXYCARBONYLATION OF ETHYLENICALLY UNSATURATED COMPOUNDS

(71) Applicant: EVONIK OPERATIONS GMBH, Essen (DE)

(72) Inventors: Carolin SCHNEIDER, Monheim am Rhein (DE); Ralf JACKSTELL, Rostock (DE); Matthias BELLER, Ostseebad Nienhagen (DE); Robert FRANKE, Marl (DE)

(73) Assignee: EVONIK OPERATIONS GMBH, Essen (DE)

(21) Appl. No.: 17/544,179

(22) Filed: Dec. 7, 2021

(30) Foreign Application Priority Data
Dec. 9, 2020 (EP) 20212753.6

(51) Int. Cl. C07F 15/00; B01J 31/24

(52) U.S. Cl. CPC C07F 15/0093; B01J 2231/321; B01J 2531/828; B01J 31/2409

(57) ABSTRACT

Platinum complexes having binaphthyl-based diphosphine ligands for the selective catalysis of the hydroxycarbonylation of ethylenically unsaturated compounds.

The present invention relates to platinum complexes having binaphthyl-based diphosphine ligands for the selective catalysis of the hydroxycarbonylation of ethylenically unsaturated compounds.

The hydroxycarbonylation of ethylenically unsaturated compounds is a process of increasing significance. A hydroxycarbonylation is understood to mean the direct reaction of ethylenically unsaturated compounds such as olefins with carbon monoxide in the presence of a metal or a metal complex and a ligand to give the corresponding acids:

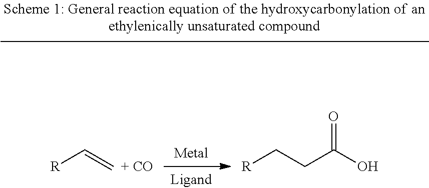

EP 3388414 A1 describes a process for hydroformylation of cyclooctadiene (COD) using 4-([1,1′:3′,1″-terphenyl]-2′-yloxy)-S-dinaphtho[2,1-d:1′,2′-f][1,3,2]dioxaphosphepine.

A disadvantage of palladium is its high cost.

The technical problem addressed by the present invention is that of providing novel complexes having a less costly metal than palladium as the central atom. The complexes are additionally to achieve good n/iso selectivities in hydroxycarbonylations.

This object is achieved by a complex according to Claim 1.

Complex comprising Pt and a compound of formula (I)

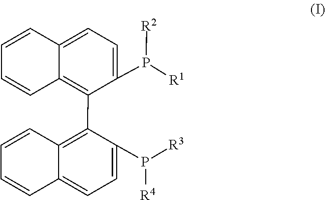

where

R¹, R², R³, R⁴ are each independently selected from —(C₁-C₁₂)-alkyl and —(C₆-C₂₀)-heteroaryl.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

The expression (C₆-C₂₀)-heteroaryl encompasses mono- or polycyclic aromatic hydrocarbyl radicals having 6 to 20 carbon atoms, where one or more of the carbon atoms are replaced by heteroatoms. Preferred heteroatoms are N, O and S. The (C₆-C₂₀)-heteroaryl groups have 6 to 20, preferably 6 to 14 and more preferably 6 to 10 ring atoms. Thus, for example, pyridyl in the context of this invention is a C₆-heteroaryl radical.

Suitable (C₆-C₂₀)-heteroaryl groups having at least six ring atoms are especially pyridyl, pyridazinyl, pyrimidyl, pyrazinyl, benzofuranyl, indolyl, isoindolyl.

In one embodiment, at least two of the R¹, R², R³, R⁴ radicals are a —(C₆-C₂₀)-heteroaryl radical having at least six ring atoms.

In one embodiment, the R¹ and R³ radicals are each a —(C₆-C₂₀)-heteroaryl radical having at least six ring atoms.

In one embodiment, the R¹ and R³ radicals are each 2-pyridyl.

In one embodiment, R² and R⁴ are —(C₁-C₁₂)-alkyl.

In one embodiment, R² and R⁴ are tert-butyl.

In one embodiment, the compound (I) has the structure (1):

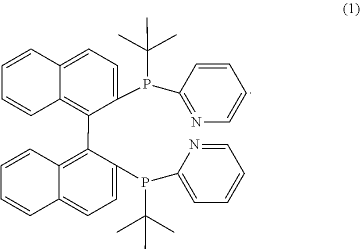 [SMILES: CC(C)(C)P(c1ccccn1)c1ccc2ccccc2c1-c1c(P(c2ccccn2)C(C)(C)C)ccc2ccccc12]

The invention further relates to the use of a complex according to the invention for catalysis of a hydroxycarbonylation reaction.

Process comprising the process steps of:

a) initially charging an ethylenically unsaturated compound;

b) adding a complex as described above, or
a compound of formula (I)

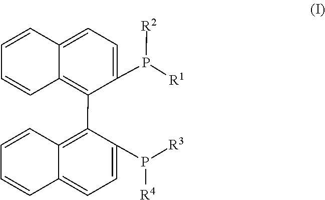

where

R¹, R², R³, R⁴ are each independently selected from —(C₁-C₁₂)-alkyl and —(C₆-C₂₀)-heteroaryl and

a substance comprising Pt;

c) adding an acid;

d) feeding in CO;

e) heating the reaction mixture from a) to d), with conversion of the ethylenically unsaturated compound to a carboxylic acid.

In this process, process steps a), b), c) and d) can be effected in any desired sequence. Typically, however, the addition of CO is effected after the co-reactants have been initially charged in steps a) to c). In addition, CO can also be fed in in two or more steps, in such a way that, for example, a portion of the CO is first fed in, then the mixture is heated, and then a further portion of CO is fed in.

The ethylenically unsaturated compounds used as reactant in the process according to the invention contain one or more carbon-carbon double bonds. These compounds are also referred to hereinafter as olefins for simplification. The double bonds may be terminal or internal.

In one variant of the process, the ethylenically unsaturated compound does not comprise any further functional groups apart from carbon-carbon double bonds.

In one variant of the process, the ethylenically unsaturated compound is selected from: ethene, propene, 1-butene, cis- and/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

In one variant of the process, the acid in process step c) is selected from: acetic acid, perchloric acid, sulfuric acid, phosphoric acid, methylphosphonic acid, methanesulfonic acid, trifluoromethanesulfonic acid, tert-butanesulfonic acid, p-toluenesulfonic acid (PTSA), 2-hydroxypropane-2-sulfonic acid, 2,4,6-trimethylbenzenesulfonic acid, dodecylsulfonic acid, camphorsulfonic acid.

In one variant of the process, the acid in process step c) is acetic acid (AcOH).

In one variant of the process, the substance comprising Pt is selected from: platinum dichloride (PtCl₂), platinum(II) acetylacetonate [Pt(acac)₂], platinum(II) acetate [Pt(OAc)₂], dichloro(1,5-cyclooctadiene)platinum(II) [Pt(cod)₂Cl₂], bis(dibenzylideneacetone)platinum [Pt(dba)₂], bis(acetonitrile)dichloroplatinum(II) [Pt(CH₃CN)₂Cl₂], (cinnamyl)platinum dichloride [Pt(cinnamyl)Cl₂].

In one variant of the process, the substance comprising Pt is selected from: platinum dichloride (PtCl₂), platinum(II) acetylacetonate [Pt(acac)₂], platinum(II) acetate [Pt(OAc)₂].

CO is fed in in step d) preferably at a partial CO pressure between 0.1 and 10 MPa (1 to 100 bar), preferably between 1 and 8 MPa (10 to 80 bar), more preferably between 2 and 6 MPa (20 to 60 bar).

The reaction mixture is heated in step e) of the process according to the invention preferably to a temperature in the range from 60° C. to 160° C., preferably in the range from 60 to 140° C., more preferably in the range from 60 to 100° C., in order to convert the ethylenically unsaturated compound to an acid.

The invention is to be illustrated in detail hereinafter by a working example.

Conversion of 1-octene to the Acid

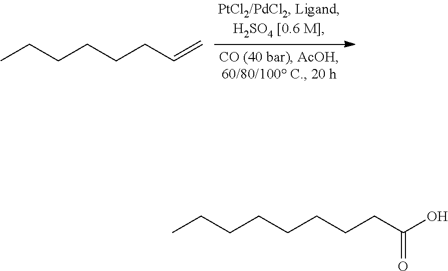

Reaction conditions: 1-octene (1.0 mmol), PtCl₂ or PdCl₂ (0.01 mmol, 1.0 mol %), ligand (1) (0.022 mmol, 2.2 mol %), sulfuric acid [0.6 M] 0.5 ml, AcOH (1.5 ml), pressure (CO): 40 bar, temperature: 60/80/100° C., reaction time: 20 h.

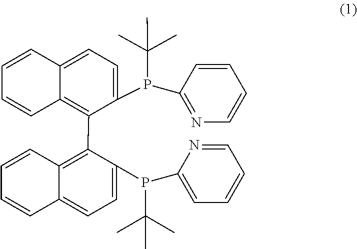 [SMILES: CC(C)(C)P(c1ccccn1)c1ccc2ccccc2c1-c1c(P(c2ccccn2)C(C)(C)C)ccc2ccccc12]

The series of experiments was conducted under analogous conditions, once with PtCl₂ and once with PdCl₂. Each series of experiments here comprises three experiments that differ merely in the temperature: 60° C., 80° C. and 100° C. The results are shown in the following table:

| Entry | Metal-Cl₂(1 mmol) | Temperature (° C.) | Selectivity (n/iso) |
|---|---|---|---|
| 1* | Pt | 60 | 94/6 |
| 2 | Pd | | 88/12 |
| 3* | Pt | 80 | 90/10 |
| 4 | Pd | | 76/24 |
| 5* | Pt | 100 | 82/18 |
| 6 | Pd | | 75/25 |

*process according to the invention

With Pt as complex metal, it was possible to achieve better n/iso selectivity at all three temperatures than with the comparative Pd complex.

The cost of Pt is below that of Pd. The object is thus achieved by a complex according to the invention.

1. Complex comprising Pt and a compound of formula (I) 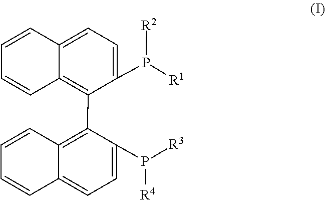
where
R¹, R², R³, R⁴ are each independently selected from —(C₁-C₁₂)-alkyl and —(C₆-C₂₀)-heteroaryl.

2. Complex according to claim 1,
wherein at least two of the R¹, R², R³, R⁴ radicals are a —(C₆-C₂₀)-heteroaryl radical having at least six ring atoms.

3. Complex according to claim 1,
wherein the R¹ and R³ radicals are each a —(C₆-C₂₀)-heteroaryl radical having at least six ring atoms.

4. Complex according to claim 1,
wherein the R¹ and R³ radicals are each 2-pyridyl.

5. Complex according to claim 1,
wherein R² and R⁴ are —(C₁-C₁₂)-alkyl.

6. Complex according to claim 1,
wherein R² and R⁴ are tert-butyl.

7. Complex according to claim 1,
wherein the compound (I) has the structure (1):

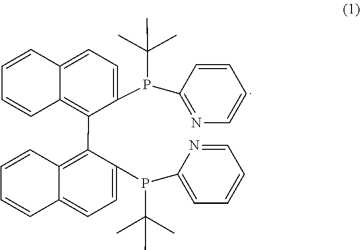 [SMILES: CC(C)(C)P(c1ccccn1)c1ccc2ccccc2c1-c1c(P(c2ccccn2)C(C)(C)C)ccc2ccccc12]

8. Process comprising the process steps of:
a) initially charging an ethylenically unsaturated compound;
b) adding a complex according to claim 1, or
a compound of formula (I) 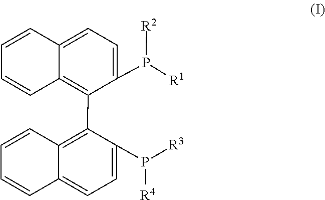
where
R¹, R², R³, R⁴ are each independently selected from —(C₁-C₁₂)-alkyl and —(C₆-C₂₀)-heteroaryl and
a substance comprising Pt;
c) adding an acid;
d) feeding in CO;
e) heating the reaction mixture from a) to d), with conversion of the ethylenically unsaturated compound to a carboxylic acid.

9. Process according to claim 8,
wherein the ethylenically unsaturated compound is selected from: ethene, propene, 1-butene, cis- and/or trans-2-butene, isobutene, 1,3-butadiene, 1-pentene, cis- and/or trans-2-pentene, 2-methyl-1-butene, 3-methyl-1-butene, 2-methyl-2-butene, hexene, tetramethylethylene, heptene, 1-octene, 2-octene, di-n-butene, or mixtures thereof.

10. Process according to claim 8,
wherein the acid in process step c) is selected from acetic acid, perchloric acid, sulfuric acid, phosphoric acid, methylphosphonic acid, methanesulfonic acid, trifluoromethanesulfonic acid, tert-butanesulfonic acid, p-toluenesulfonic acid, 2-hydroxypropane-2-sulfonic acid, 2,4,6-trimethylbenzenesulfonic acid, dodecylsulfonic acid, camphorsulfonic acid.

11. Process according to claim 8,
wherein the acid in process step c) is acetic acid.

12. Process according to claim 8,
wherein the substance comprising Pt is selected from: platinum dichloride (PtCl₂), platinum(II) acetylacetonate [Pt(acac)₂], platinum(II) acetate [Pt(OAc)₂], dichloro(1,5-cyclooctadiene)platinum(II) [Pt(cod)₂Cl₂], bis(dibenzylideneacetone)platinum [Pt(dba)₂], bis(acetonitrile)dichloroplatinum(II) [Pt(CH₃CN)₂Cl₂], (cinnamyl)platinum dichloride [Pt(cinnamyl)Cl₂].

13. Process according to claim 8,
wherein the substance comprising Pt is selected from: platinum dichloride (PtCl₂), platinum(II) acetylacetonate [Pt(acac)₂], platinum(II) acetate [Pt(OAc)₂].
